(19) United States
(12) Patent Application Publication
(10) Pub. No.: US 2017/0349858 A1
(43) Pub. Date: Dec. 7, 2017

(54) PHOTOLABILE FRAGRANCE PRECURSOR COMPOUND

(71) Applicant: Henkel AG & Co. KGaA, Duesseldorf (DE)

(72) Inventors: Christian Kropf, Hilden (DE); Ursula Huchel, Cologne (DE); Nadine Bluhm, Duesseldorf (DE); Andreas Bauer, Kaarst (DE)

(21) Appl. No.: 15/537,446

(22) PCT Filed: Dec. 8, 2015
(86) PCT No.: PCT/EP2015/078982

§ 371 Date: Jun. 19, 2017

(30) Foreign Application Priority Data
Dec. 17, 2014 (DE) 10 2014 226 196.1

(51) Int. Cl. C11B 9/00; C11D 3/50; A61Q 19/00; A61K 8/35; A61L 9/18; A61L 9/00; C11D 11/00; C07C 49/84

(52) U.S. Cl. CPC C11B 9/0003; C11D 11/007; C11D 3/50; C11B 9/0061; C07C 49/84; A61K 8/35; A61L 9/18; A61L 9/00; A61Q 19/00; A61K 2800/10; A61K 2800/81

(57) ABSTRACT

The invention relates to specific phenacyl ethers of formula (I), as defined herein, which can be used as photolabile pro-fragrances. The invention further relates to washing and cleaning agents, cosmetic agents, and air freshening agents containing such phenacyl ethers, and a method for the long-lasting scenting of surfaces.

### CROSS-REFERENCE TO RELATED APPLICATIONS

The present application is a national stage entry according to 35 U.S.C. §371 of PCT Application No. PCT/EP2015/078982 filed on Dec. 8, 2015, which claims priority to German Patent Application No. 102014226196.1, filed on Dec. 17, 2014; both of which are herein incorporated by reference in their entirety.

### TECHNICAL FIELD

The subject matter herein generally relates to the field of pro-fragrances, as used, for example, in the area of washing or cleaning agents, cosmetic agents, and air freshening agents; and more specifically to phenacyl ethers that function as photolabile pro-fragrances.

### BACKGROUND

Washing and cleaning agents and cosmetic agents generally contain fragrances that impart a pleasant odor. The fragrances typically mask the odor of other ingredients, so that the user has the impression of an appealing odor.

Fragrances, in particular in the area of washing agents, are important components in the formulation, since it is desirable for laundry to have a pleasant, fresh fragrance in both the wet and dry states. A basic problem associated with the use of fragrances is that they are more or less highly volatile compounds, although a long-lasting fragrance effect is desired. In particular for odorants that represent the fresh, light fragrance notes of the perfume, but evaporate particularly quickly due to their relatively high vapor pressure, it is difficult to achieve the desired long-lasting fragrance impression. Photolabile pro-fragrance molecules represent one option for the delayed release of fragrances. The effect of sunlight or some other source of electromagnetic radiation of a certain wavelength induces splitting of a covalent bond in the pro-fragrance molecule, thereby releasing a fragrance.

U.S. Pat. No. 6,949,680, for example, discloses the use of certain phenyl or pyridyl ketones as photoactivatable substances, which in the presence of light release a terminal alkene as an active substance in a photochemical fragmentation process. This active substance has fragrance-imparting or antimicrobial activity, for example, that is initially delayed due to the photochemically induced decomposition, and released on an intended surface over an extended period of time.

International Patent publication WO 2009/118219 A1 discloses photoactivatable substances that enable release of cyclic terpenes or cyclic terpenoids, and WO 2011/101180 discloses the use of certain ketones as photoactivatable substances that release an active substance in the presence of light in a photochemical fragmentation process. This active substance has fragrance-imparting activity, for example, that is initially delayed due to the photochemically induced decomposition, and released on an intended surface over an extended period of time.

However, when known pro-fragrance molecules are used, the fragrance intensity is low and the fragrance effect has only a short duration. In addition, conventional pro-fragrance molecules result in noticeable yellowing after the fragrance is released, which is undesirable. Therefore, there is a need for pro-fragrance molecules that effectively release fragrances and do not become discolored.

### SUMMARY

According to a non-limiting embodiment, a compound having formula (I) is presented, i.e.:

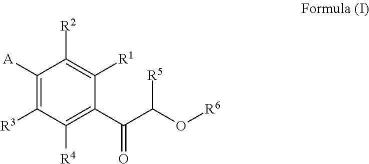

where
A is selected from the group consisting of hydrogen; substituted or unsubstituted linear or branched alkyl, alkenyl, or alkynyl having up to 20; carbon atoms, substituted or unsubstituted linear or branched heteroalkyl, heteroalkenyl, or heteroalkynyl having up to 20 carbon atoms, and from 1 to 6 heteroatoms selected from the group consisting of O, S, and N; substituted or unsubstituted aryl having up to 20 carbon atoms; substituted or unsubstituted heteroaryl having up to 20 carbon atoms, and from 1 to 6 heteroatoms selected from the group consisting of O, S, and N; cycloalkyl or cycloalkenyl having up to 20 carbon atoms; and heterocycloalkyl or heterocycloalkenyl having up to 20 carbon atoms, and from 1 to 6 heteroatoms selected from the group consisting of O, S, and N;
R¹, R², R³, R⁴, and R⁵ are independently selected from the group consisting of hydrogen; —OH; —NH₂; halogen; substituted or unsubstituted linear or branched alkyl, alkenyl, or alkynyl having up to 20 carbon atoms; substituted or unsubstituted linear or branched heteroalkyl, heteroalkenyl, or heteroalkynyl having up to 20 carbon atoms, and from 1 to 6 heteroatoms selected from the group consisting of O, S, and N; substituted or unsubstituted aryl having up to 20 carbon atoms; substituted or unsubstituted heteroaryl having up to 20 carbon atoms, and from 1 to 6 heteroatoms selected from the group consisting of O, S, and N; cycloalkyl or cycloalkenyl having up to 20 carbon atoms; and heterocycloalkyl or heterocycloalkenyl having up to 20 carbon atoms, and from 1 to 6 heteroatoms selected from the group consisting of O, S, and N; and
R⁶ is a linear, branched, or cyclic, substituted or unsubstituted hydrocarbon functional group having from 6 to 30 carbon atoms and from 0 to 10 heteroatoms selected from the group consisting of N, O, S, and Si.

According to another non-limiting embodiment, a composition may include the compound where the composition may be, but is not limited to, a washing agent, a cleaning agent, an air freshening agent, a cosmetic agent, and combinations thereof.

According to yet another non-limiting embodiment of a method, a method may include applying at least one compound of Formula (I) to at least one surface to be scented and exposing the surface(s) to electromagnetic radiation having a wavelength ranging from about 200 to about 600 nm.

### DETAILED DESCRIPTION

It has now surprisingly been found that specific phenacyl ethers of formula (I) are suitable for releasing fragrances over a long period of time without becoming discolored. This results in an attractive appearance of the article, for example textiles, to which the compound has been applied. In addition, the compounds may release fragrances over a long period of time and thus ensure a long-lasting fragrance effect, in particular in conjunction with the textile treatment. For example, when the phenacyl ethers described herein are used in a laundry treatment agent, i.e., a washing agent or softener, it is possible to achieve an improved long-term fragrance effect for the treated laundry. The compounds may also allow the total quantity of fragrance contained in the agent to be reduced, while still achieving fragrance benefits on the laundered textiles, in particular with regard to the feeling of freshness.

In a first non-limiting aspect, compounds of formula (I) may be suitable for releasing fragrances over a long period of time without becoming discolored. Formula (I) may have the following formula:

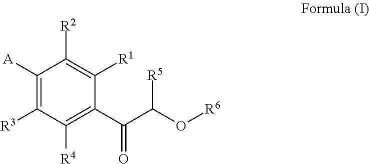

where
A is selected from the group comprising hydrogen; substituted or unsubstituted linear or branched alkyl, alkenyl, or alkynyl having up to 20, alternatively up to 12, carbon atoms; substituted or unsubstituted linear or branched heteroalkyl, heteroalkenyl, or heteroalkynyl having up to 20, alternatively up to 12, carbon atoms, and from 1 to 6, alternatively from 1 to 4, heteroatoms selected from O, S, and N; substituted or unsubstituted aryl having up to 20, alternatively up to 12, carbon atoms; substituted or unsubstituted heteroaryl having up to 20, alternatively up to 12, carbon atoms, and from 1 to 6, alternatively 1 to 4, heteroatoms selected from O, S, and N; cycloalkyl or cycloalkenyl having up to 20, alternatively up to 12, carbon atoms; and heterocycloalkyl or heterocycloalkenyl having up to 20, alternatively up to 12, carbon atoms, and from 1 to 6, alternatively from 1 to 4, heteroatoms selected from O, S, and N;
R¹, R², R³, R⁴, and R⁵ in each case are independently selected from the group comprising hydrogen; —OH; —NH₂; halogen; substituted or unsubstituted linear or branched alkyl, alkenyl, or alkynyl having up to 20, alternatively up to 12, carbon atoms, substituted or unsubstituted linear or branched heteroalkyl, heteroalkenyl, or heteroalkynyl having up to 20, alternatively up to 12, carbon atoms, and from 1 to 6, alternatively from 1 to 4, heteroatoms selected from O, S, and N; substituted or unsubstituted aryl having up to 20 alternatively up to 12, carbon atoms substituted or unsubstituted heteroaryl having up to 20, alternatively up to 12, carbon atoms, and from 1 to 6, alternatively 1 to 4, heteroatoms selected from O, S, and N; cycloalkyl or cycloalkenyl having up to 20, alternatively up to 12, carbon atoms and heterocycloalkyl or heterocycloalkenyl having up to 20, alternatively up to 12, carbon atoms, and from 1 to 6, alternatively 1 to 4, heteroatoms selected from O, S, and N; and
R⁶ may be a linear, branched, or cyclic, substituted or unsubstituted hydrocarbon functional group having from 6 to 30, alternatively 6 to 20, carbon atoms and from 0 to 10 heteroatoms selected from N, O, S, and Si.

In another aspect, the compound may be or include, but is not limited to a washing agent, a cleaning agent, an air freshening agent, a cosmetic agent, and combinations thereof where the compound comprises at least one compound of formula (I).

Lastly, the present compound may be incorporated into a method for the long-lasting scenting of surfaces, in which the compound may be applied to the surface(s) to be scented, and the surface(s) is subsequently exposed to electromagnetic radiation to a wavelength ranging in length from about 200 to about 600 nm.

“At least one,” as used herein, refers to 1 or more, for example 2, 3, 4, 5, 6, 7, 8, 9 or more. In conjunction with components of the compound described herein, this statement refers not to the absolute quantity of molecules, but, rather, to the type of component. “At least one compound of formula (I)” therefore means, for example, one or more different compounds of formula (I), i.e., one or more different types of compounds of formula (I). In the context of stated quantities, the stated quantities refer to the total quantity of the correspondingly designated type of component, as defined above.

“Alkyl,” as used herein, refers to a saturated aliphatic hydrocarbon, including straight-chain and branched-chain groups. The alkyl group may have from 1 to 10 carbon atoms (when a numerical range “1 to 10,” for example, is stated herein, this means that this group, in the present case the alkyl group, may have 1 carbon atom, 2 carbon atoms, 3 carbon atoms, etc., up to and including 10 carbon atoms). In particular, the alkyl may be a medium alkyl, having from 1 to 6 carbon atoms, or a low alkyl, having from 1 to 4 carbon atoms, for example methyl, ethyl, n-propyl, isopropyl, butyl, isobutyl, tert-butyl, etc.

“Alkenyl” refers to an alkyl group, as defined herein, comprising at least two carbon atoms and at least one carbon-carbon double bond, such as but not limited to, ethenyl, propenyl, butenyl, or pentenyl and the structural isomers thereof such as 1- or 2-propenyl, 1-, 2-, or 3-butenyl, etc.

“Alkynyl” refers to an alkyl group, as defined herein, made up of at least two carbon atoms and at least one carbon-carbon triple bond, such as but not limited to, ethynyl (acetylene), propynyl, butynyl, or pentynyl, and the structural isomers thereof as described above.

“Heteroalkyl,” “heteroalkenyl,” and “heteroalkynyl,” as used herein, refer to alkyl, alkenyl, and alkynyl groups, respectively, as defined above, in which one or more carbon atoms are replaced by heteroatoms, such as, but not limited to, O, S, N, and Si, such as, but not limited to, ethoxyethyl, ethoxyethenyl, isopentoxypropyl, and alkoxy functional groups of formula —O-alkyl, such as methoxy, ethoxy, propoxy, etc.

A “cycloalkyl” group refers to monocyclic or polycyclic (multiple rings having shared carbon atoms) groups, comprising from 3 to 8 carbon atoms, in which the ring does not have a complete conjugated pi electron system, such as but not limited to, cyclopropyl, cyclobutyl, cyclopentyl, cyclohexyl, cyclobutenyl, cyclopentenyl, cyclohexenyl, etc. Examples of cycloalkyl groups are cyclopropane, cyclobutane, cyclopentane, cyclopentene, cyclohexane, adamantane, cyclohexadiene, cycloheptane, and cycloheptatriene.

“Aryl” refers to monocyclic or polycyclic (i.e., rings having shared neighboring carbon atom pairs) groups comprising from 6 to 14 carbon ring atoms that have a complete conjugated pi electron system. Examples of aryl groups may be or include, but are not limited to phenyl, naphthalenyl, and anthracenyl.

A “heteroaryl” group refers to a monocyclic or polycyclic (i.e., rings that share a neighboring ring atom pair) aromatic ring comprising from 5 to 10 ring atoms, wherein one, two, three, or four ring atoms are nitrogen, oxygen, or sulfur and the remainder are carbon. Non-limiting examples may be or include, but are not limited to, heteroaryl groups comprising pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-triazinyl, 1,2,3-triazinyl, benzofuryl, isobenzofuryl, benzothienyl, benzotriazolyl, isobenzothienyl, indolyl, isoindolyl, 3H-indolyl, benzimidazolyl, benzothiazolyl, benzoxazolyl, quinolizinyl, quinazolinyl, pthalazinyl, quinoxalinyl, cinnolinyl, naphthyridinyl, quinolyl, isoquinolyl, tetrazolyl, 5,6,7,8-tetrahydroquinolyl, 5,6,7,8-tetrahydroisoquinolyl, purinyl, pteridinyl, pyridinyl, pyrimidinyl, carbazolyl, xanthenyl, or benzoquinolyl.

A “heterocycloalkyl” group refers to a monocyclic or fused ring, comprising from 5 to 10 ring atoms, comprising one, two, or three heteroatoms such as, but not limited to, N, O, and S, the remainder of the ring atoms being carbon. A “heterocycloalkenyl” group additionally comprising one or more double bonds. However, the ring may not have a complete conjugated pi electron system. Non-limiting examples may be or include, but are not limited to, heteroalicyclic groups comprising pyrrolidine, piperidine, piperazine, morpholine, imidazolidine, tetrahydropyridazine, tetrahydrofuran, thiomorpholine, tetrahydropyridine, and the like.

“Substituted,” as used herein in conjunction with the substituents and functional groups is defined herein to mean that one or more H atoms are replaced by other functional groups for the groups in question, these functional groups being selected in particular from those containing one or more heteroatoms. In various embodiments, the substituents may be or include, but are not limited to, ═O, ═S, —OH, —SH, —NH₂—NO₂, —CN, —F, —Cl, —Br, C₁₋₁₀ alkyl, C₂₋₁₀ alkenyl, C₂₋₁₀ alkynyl, C₃₋₈ cycloalkyl, C₆₋₁₄ aryl, a 5- to 10-membered heteroaryl ring in which 1 to 4 ring atoms are independently nitrogen, oxygen, or sulfur; and a 5- to 10-membered heteroalicyclic ring in which 1 to 3 ring atoms are independently nitrogen, oxygen, or sulfur.

In various embodiments of the present invention, A is hydrogen or a linear alkyl having 1 to 3 carbon atoms. Alternatively, A is hydrogen or a methyl group, and in yet another non-limiting embodiment, A is a methyl group.

According to a non-limiting embodiment, A is neither hydroxyl (—OH) nor an alkoxy group. Namely, it has been found that yellowing after release of the fragrance may be prevented, and a long-lasting fragrance release achieved, by appropriate other substituents at this position in the benzene ring, and by the specific structure of the compound of formula (I).

In various embodiments, R¹, R⁴, and R⁵, independently of one another, are in each case hydrogen or methyl groups, alternatively with R¹ and R⁴ being hydrogen and R⁵ being a methyl group. In other embodiments, R² and R³, independently of one another, are in each case hydrogen or a substituted or unsubstituted, linear or branched alkyl having 1 to 5 carbon atoms. In one non-limiting embodiment, R² and R³, independently of one another, are in each case hydrogen or tert-butyl, or alternatively hydrogen.

In the compounds of formula (I), R⁶ is a linear, branched, or cyclic, substituted or unsubstituted hydrocarbon functional group having from 6 to 30, alternatively from 6 to 20, carbon atoms and from 0 to 10 heteroatoms such as, but not limited to, N, O, S, and Si.

In one non-limiting embodiment, R⁶ is a substituted or unsubstituted linear or branched alkyl having from 6 to 20, alternatively from 6 to 15, carbon atoms; a substituted or unsubstituted linear or branched alkenyl having 6 to 20, alternatively from 6 to 15, carbon atoms; a substituted or unsubstituted alkylaryl having 6 to 20, alternatively from 6 to 15, carbon atoms; a substituted or unsubstituted aryl having from 6 to 20, alternatively from 6 to 15, carbon atoms; a cycloalkyl having from 6 to 20, alternatively from 6 to 15, carbon atoms; and a cycloalkenyl having from 6 to 20, alternatively 6 to 15, carbon atoms.

The compound of formula (I) according to the invention is suitable as a pro-fragrance for all fragrance aldehydes and fragrance ketones that are known in the prior art and suited for this purpose. Therefore, in various embodiments R⁶ is a functional group that is derived from a fragrance aldehyde or fragrance ketone. “Derived functional group,” as used in this context, refers to functional groups R⁶ that are derived from fragrance aldehydes or fragrance ketones for which the oxygen atom of the compound of formula (I), to which R⁶ is bound, is/was the aldehyde oxygen or keto oxygen of the fragrance aldehyde or fragrance ketone, respectively. When the fragrance is released, the bond between the oxygen atom, to which R⁶ is bound, and the carbon atom, which bears R⁵, is cleaved, resulting in the fragrance aldehyde or fragrance ketone and the corresponding (optionally substituted) phenacyl functional group as cleavage products.

The fragrance aldehydes and fragrance ketones, which as a functional group R⁶ are bound to the phenacyl structure according to formula (I) via their aldehyde oxygen or keto oxygen atom, respectively, and are released by cleavage, in various embodiments are selected from the group comprising geranial; neral; (R)-citronellal; (S)-citronellal; buccoxime; isojasmone; methyl beta-naphthyl ketone; musk indanone; Tonalid/Musk Plus; alpha-damascone, beta-damascone, delta-damascone, gamma-damascone, damascenone, damarose, methyl dihydrojasmonate, menthone, carvone, hedione, camphor, fenchone, alpha-ionone, beta-ionone, gamma-methylionone, fleuramone, dihydrojasmone, cis-jasmone, Iso E Super, methyl cedrenyl ketone, methyl cedrylone, acetophenone, methylacetophenone, para-methoxyacetophenone, benzylacetone, benzophenone, para-hydroxyphenylbutanone, celery ketone, livescone, 6-isopropyldecahydro-2-naphtone, dimethyl octenone, Frescomenthe, 4-(1-ethoxyvinyl)-3,3,5,5-tetramethylcyclohexanone, methyl heptenone, 2-(2-(4-methyl-3-cyclohexen-1-yl)propyl)cyclopentanone, 1-(p-menthen-6(2)yl)-1-propanone, 4-(4-hydroxy-3-methoxyphenyl)-2-butanone, 2-acetyl-3,3-dimethylnorbomane, 6,7-dihydro-1,1,2,3,3-pentamethyl-4(5h)indanone, 4-damascol, dulcinyl, Cassione, gelsone, hexalone, Isocyclemone E, methyl cyclocitrone, methyl lavender ketone, orivone, para-tertiary butylcyclohexanone, verdone, Delphone, muscone, neobutenone, Plicatone, Veloutone, 2,4,4,7-tetramethyloct-6-en-3-one, and tetramerane.

In general, the following are suited as fragrance ketones, without limiting the compound thereto:

4-methoxyphenyl ethanone; 1-[6-(1,1-dimethylethyl)-2,3-dihydro-1,1-dimethyl-1H-inden-4-yl]ethanone; 1-(5,6,7,8-tetrahydro-3,5,5,6,8,8-hexamethyl-2-naphthalenyl)ethanone; 2-(1-methylethyl)indanone; 4-tert-butyl-3,5-dinitro-2,6-dimethylacetophenone; 1,6,7,8-tetrahydro-1,4,6,6,8,8-hexamethyl-as-indacen-3(2H)one; 1-(2-naphthalenyl)ethanone; 1-(2,3-dihydro-1,1,2,3,3,6-hexamethyl-1H-inden-5-yl)ethanone; 1-[2,3-dihydro-1,1,2,6-tetramethyl-3-(1-methylethyl)-1H-inden-5-yl]ethanone; 3-methyl-1-(4-methylphenyl)-4-hexen-1-one; 5-acetyl-1,1,2,3,3-pentamethylindane; 1-phenylpropanone; acetophenone; 2,4-dimethylphenylethanone; 1-[4-(1,1-dimethylethyl)-2,6-dimethylphenyl]ethanone; 1-(hexahydrodimethyl-1H-benzindenyl)ethanone; 1-(5,6,7,8-tetrahydro-2-naphthalenyl)ethanone; 1-phenyl-4-penten-1-one; 1-(5,6,7,8-tetrahydro-3,5,5,8,8-pentamethyl-2-naphthalenyl)ethanone; 1-(3-ethyl-5,6,7,8-tetrahydro-5,5,8,8-tetramethyl-2-naphthalenyl)ethanone; 2,5-dimethyl-oct-2-en-6-one; 4-(2,6,6-trimethylcyclohex-1-en-1-yl)-butan-2-one; 4-(2,6,6-trimethylcyclohex-2-en-1-yl)butan-2-one; 2-methyl-5-(1-methylethenyl)cyclohex-2-en-1-one; 1-(4-hydroxyphenyl)butan-3-one; 4-benzo-1,3-dioxo-5-yl-but-2-one; 2-heptylcyclopentanon-nonan-2-one; octan-2-one; 2,2,6,10-tetrametyltricyclo-[5.4.0.0(6,10)]undecan-4-one; heptan-2-one; undecan-2-one; benzylacetone; butan-2-one; 1,2,3,5,6,7-hexahydro-1,1,2,3,3-pentamethyl-4H-inden-4-one; 6-methylhept-5-en-2-one; 2-(butan-2-yl)cyclohexanone; 2-hexylcyclopent-2-en-1-one; 2-(1-methylethyl)-5-methyl-cyclohexanone; 2-(2-methylethyl)-5-methylcyclohexanone; 3-methylcyclopentadecanone; 4-(1,1-dimethylpropyl)cyclohexanone; 6,10-dimethylundeca-5,9-dien-2-one; 3-oxo-2-pentylcyclopentane acetic acid methyl ester; 1-(1,2,3,4,5,6,7,8-octahydro-2,3,8,8-tetramethyl-2-naphthalenyl)ethanone; 3-methyl-5-propylcyclohex-2-en-1-one; 1-(2-cyclohexene)-2,4,4-trimethyl-but-2-enone; carvone; 2-hexylcyclopent-2-en-1-one; 2-pentylcyclopent-2-en-1-one; 3-methyl-2-pentylcyclopent-2-en-1-one; 2-hexylidenecyclopentanone; 3,5-diethyl-5,6-dimethyl-2-cyclohexenone; 4,4a,5,6,7,8-hexahydro-6-isopropenyl-4,4a-dimethyl-2(3H)naphthalenone; 3-methyl-6-propylidenecyclohexanone; 4-(1-methylethyl)cyclohex-2-en-1-one; (E)-oct-3-en-2-one; 1-(2,3,4,7,8,8A-hexahydro-3,6,8, 8-tetramethyl-1H-3a,7-methanoazulen-5-yl)ethanone; 2-hydroxy-3,5-dimethylcyclopent-2-en-1-one; 1-(3,3-dimethyl-1-cyclohexen-1-yl)ethanone; 1-(2,4,6-trimethylcyclohex-3-en-1-yl)but-1-en-3-one; acetylisolongifolen; 2-(3-methylbut-2-en-1-yl)-3-methylcyclopent-2-en-1-one; 3-methyl-5-(2,2,3-trimethylcyclopent-3-en-1-yl)pent-3-en-2-one; 5-butylidene-2,2,4-trimethylcyclopentanone; 1,2,3,5,6,7-hexahydro-1,1,2,3,3-pentamethyl-4H-inden-4-one; 3-methyl-5-propylcyclohex-2-en-1-one; 4,4a,5,6,7,8-hexahydro-6-isopropyl-2(3H)naphthalenone; 3,5,5-trimethylcyclohex-2-en-1,4-dione; (E)-5-methyl-2-hepten-4-one; acetyldiisoamylene; dec-3-en-2-one; 2-ethyl-3,6,6-trimethylcyclohex-2-enyl-but-2-en-1-one; 1-(5,5-dimethyl-1(6)-cyclohexen-1-yl)-4-penten-1-one; 1-(2,6,6-trimethyl-1-cyclohexen-1-yl)but-2-en-1-one; 1-(2,6,6-trimethyl-2-cyclohexen-1-yl)but-2-en-1-one; 1-(2,6,6-trimethyl-3-cyclohexen-1-yl)but-2-en-1-one; 2,4,4,5,5-pentamethyl-1-cyclopenten-1-yl-ethanone; and combinations thereof.

In general, the following are suited as fragrance aldehydes, without limiting the compound thereto:

2,6,10-trimethylundec-9-enal; 1,2,3,4,5,6,7,8-octahydro-8,8-dimethyl-2-naphthalene-carboxaldehyde; tridecanal; 2-[4-(1-methylethyl)phenyl]ethanal; 2,4-dimethylcyclohex-3-en-1-carboxaldehyde; 4-carboxaldehyde-1,3,5-trimethylcyclohex-1-ene; 1-carboxaldehyde-2,4-dimethylcyclohex-3-ene; 1-carboxaldehyde-4-(4-hydroxy-4-methylpentyl)cyclohex-3-ene; hex-2-enal; 3,5,5-trimethylhexanal; heptanal; 2,6-dimethylhept-5-enal; decanal; dec-9-enal; dec-4-en-1-al; 2-methyldecanal; undec-10-en-1-al; undecanal; dodecanal; 2-methylundecanal; 2-tridecenal; octanal; nonanal; 3,5,5-trimethylhexanal; 2-nonenal; undec-9-enal; 2-phenylpropanal; 4-methylphenylacetaldehyde; 3,7-dimethyloctanal; dihydrofarnesal; 7-hydroxy-3,7-dimethyloctanal; 2,6-dimethyl-oct-5-en-1-al; 2-(4-(1-methylethyl)phenyl)ethanal; 3-(3-isopropylphenyl)butanal; 2-(3,7-dimethyloct-6-en-oxy)ethanal; 1-carboxaldehyde-4-(4-methyl-3-penten-1-yl)cyclohex-3-ene; 2,3,5,5,-tetramethylhexanal; longifolia aldehyde; 2-methyl-4-(2,6,6-trimethylcyclohex-2-en-1-yl)butanal; 2-methyl-3-(4-tert-butylphenyl)propanal; 4-(1,1-dimethylethyl)benzenepropanal; 2-[4-(1-methylethyl)phenyl]propanal; alpha-methyl-1,3-benzodioxol-5-propanal; 3,7-dimethyloct-6-en-1-al; 2-methyl-3-(p-isopropylphenyl)propionaldehyde; 4-(4-hydroxy-4-methylpentyl)cyclohex-3-en-1-carboxaldehyde; alpha-methyl-1,3-benzodioxol-5-propanal; 1-carboxaldehyde-4-(1,1-dimethylethyl)cyclohexane; 4-(octahydro-4,7-methano-5H-inden-5-ylidene)butanal; [(3,7-dimethyl-6-octenyl)oxy]acetaldehyde; 3,7-dimethyloct-2,6-dien-1-al; nonadienal; 2,4-dimethyl-2,6-heptadienal; trans-dec-2-en-1-al; 2,4-diethylhep-2,6-dien-1-al; dodec-2-en-1-al; 3,7-dimethyloct-2,6-dien-1-al; 2,4-diethylhepta-2,6-dienal; 3,7-dimethylnona-2,6-dien-1-al; 3-propyl-2-hepten-1-al; 1-carboxaldehyde-4-(prop-2-en-2-yl)cyclohex-1-ene; and combinations thereof.

In various embodiments, the compound according may be a compound according to one of the following formulas (II) through (XXV):

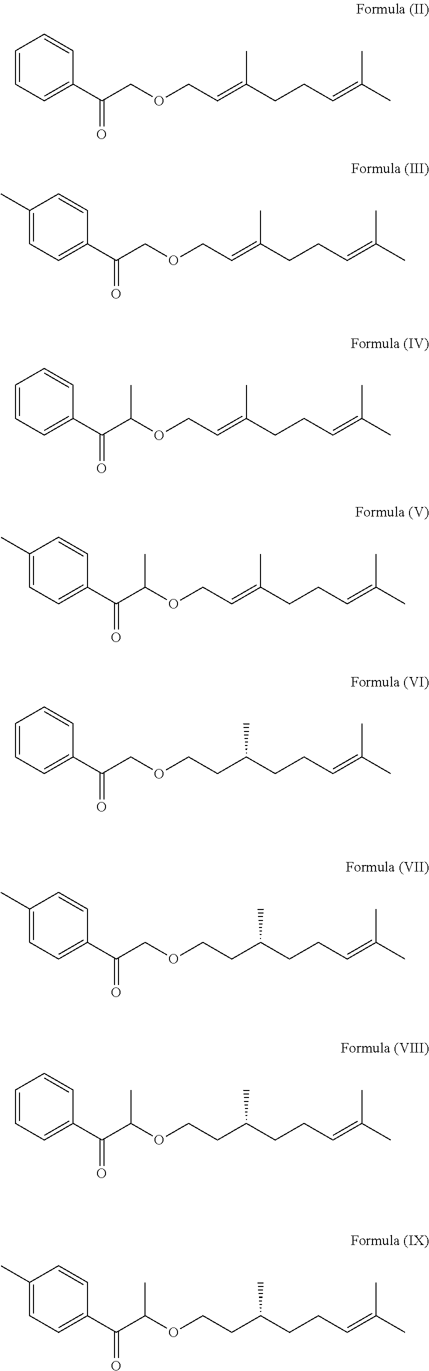

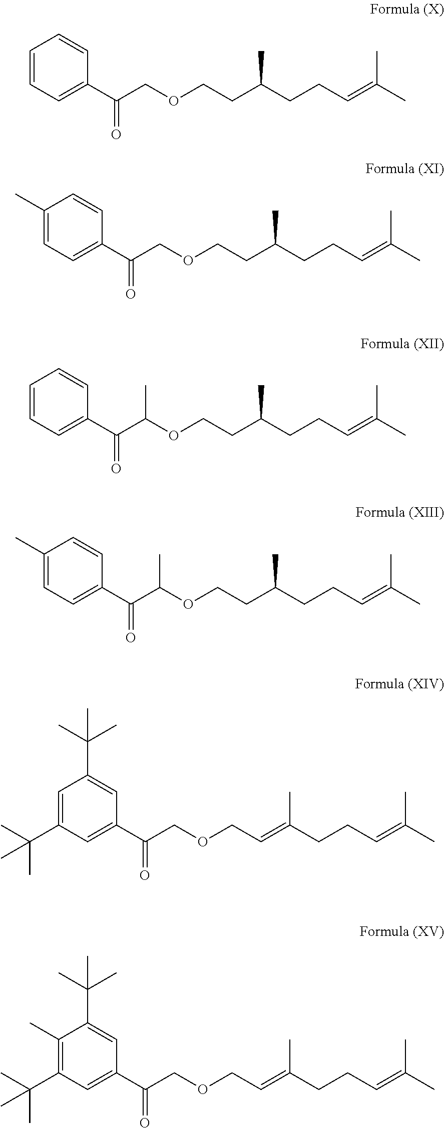

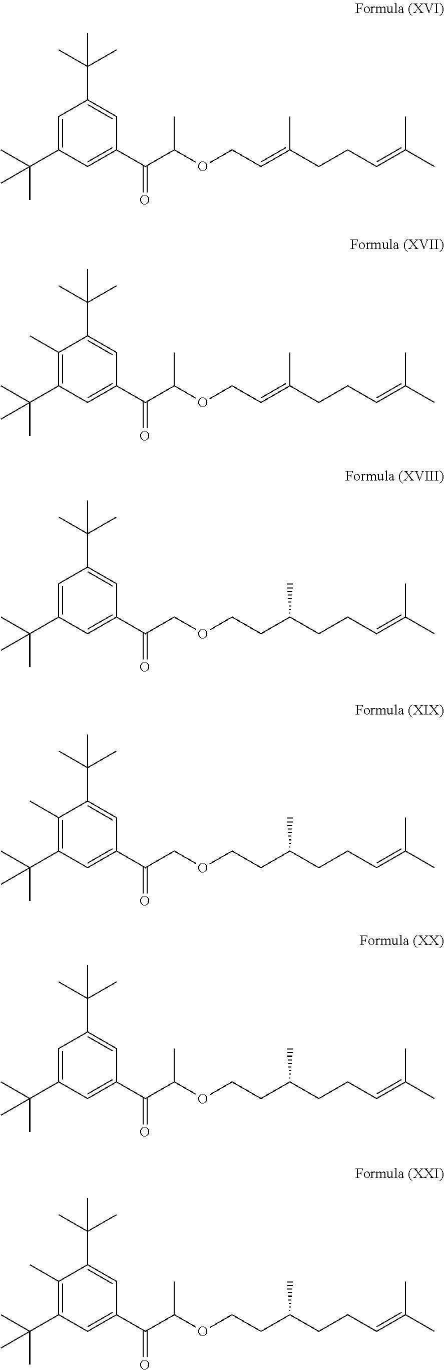

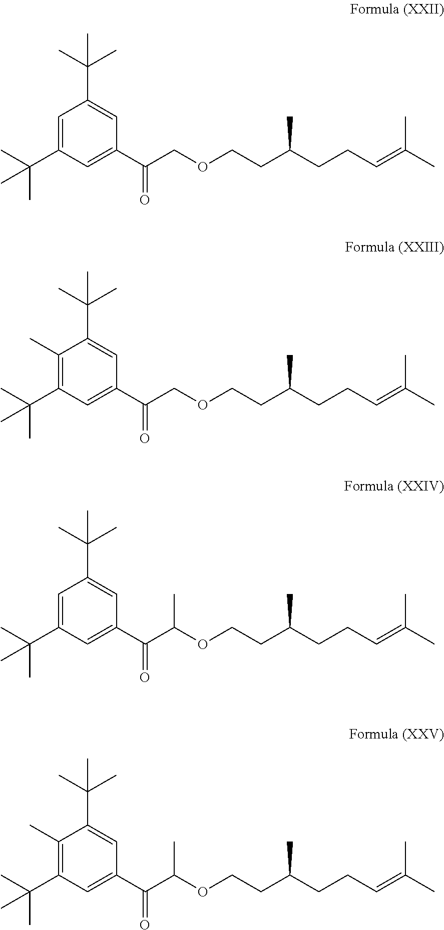

However, the compounds (II) through (XXV) may also be individually used as mixtures. For example, the respective racemic mixtures may also be used, such as a mixture of compounds (IV) and (VI) or a mixture of compounds (V) and (VII).

The compounds may be stably incorporated into the customary washing or cleaning agent matrices, cosmetics, and existing odorant compositions. They enable a delayed release of the stored fragrance ketones and fragrance aldehydes. Non-limiting fragrances may be or include but are not limited to, geranial, neral, (R)-citronellal, and (S)-citronellal. These fragrances impart a particularly long-lasting impression of freshness to washing or cleaning agents and cosmetics. In particular the dried, washed textile benefits from the good long-term fresh scent effect. The slow release of the stored odorant takes place after action by light (electromagnetic radiation) of wavelength having a length ranging from about 200 to about 600 nm.

The compound may be present in an amount ranging from about 0.0001 to about 5% by weight, alternatively from about 0.001 to about 4% by weight or from about 0.005 to about 3% by weight, or from about 0.01 to about 2% by weight, in each case based on the total weight of the agent. Examples of suitable cleaning agents are cleaning agents for hard surfaces, in particular dishwashing detergents. The cleaning agents may also be, for example, household cleaners, all-purpose cleaners, window cleaners, floor cleaners, etc. In various embodiments, this may involve a product for cleaning toilet bowls and urinals, in particular a flush cleaner for hanging in the toilet bowl.

According to a non-limiting embodiment, the washing or cleaning agent includes at least one surfactant, such as but not limited to, an anionic surfactant, a cationic surfactant, a nonionic surfactant, a zwitterionic surfactant, an amphoteric surfactant, or mixtures thereof.

According to another a non-limiting embodiment, the agent may be present in solid or liquid form.

A further non-limiting embodiment of the compound relates to a cosmetic agent, comprising at least one compound according to formula (I), where the compound may be present in an amount ranging from about 0.0001 to about 50% by weight, alternatively from about 0.001 to about 5% by weight, or from about 0.005 to about 3% by weight, or from about 0.01 to about 2% by weight, in each case based on the overall agent.

A further non-limiting embodiment relates to an air freshening agent (for example, room freshener, room deodorizer, room spray, etc.) comprising at least one compound according to formula (I), wherein the compound of formula (I) is present in an amount ranging from about 0.0001 to about 50% by weight, alternatively from about 0.001 to about 5% by weight, alternatively from about 0.01 to about 3% by weight, or from about 0.1 to about 2% by weight, in each case based on the total weight of the agent.

According to another non-limiting embodiment, additional fragrances are contained in an agent according to the invention (i.e., washing or cleaning agent, cosmetic agent, or air freshening agent), such as fragrances of natural or synthetic origin, e.g. lower-volatility fragrances, higher-boiling fragrances, solid fragrances, and/or semisolid fragrances.

Examples of semisolid odorants that are usable within the scope of the present invention are essential oils such as angelica root oil, anise oil, arnica blossom oil, basil oil, bay oil, bergamot oil, champaca blossom oil, abies alba oil, abies alba cone oil, elemi oil, eucalyptus oil, fennel oil, spruce needle oil, galbanum oil, geranium oil, ginger grass oil, guaiac wood oil, gurjun balsam oil, helichrysum oil, ho oil, ginger oil, iris oil, cajeput oil, calamus oil, chamomile oil, camphor oil, cananga oil, cardamom oil, cassia oil, pine needle oil, copaiba balsam oil, coriander oil, spearmint oil, caraway oil, cumin oil, lavender oil, lemongrass oil, lime oil, mandarin oil, melissa oil, musk seed oil, myrrh oil, clove oil, neroli oil, niaouli oil, olibanum oil, orange oil, oregano oil, palmarosa oil, patchouli oil, balsam Peru oil, petitgrain oil, pepper oil, peppermint oil, allspice oil, pine oil, rose oil, rosemary oil, sandalwood oil, celery oil, spike lavender oil, star anise oil, turpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, citronella oil, lemon oil, and cypress oil.

However, higher-boiling and solid odorants of natural or synthetic origin may also be used within the scope of the present invention as semisolid odorants or odorant mixtures, i.e., fragrances. These include the following compounds and mixtures thereof: ambrettolid, alpha-amylcinnamaldehyde, anethole, anisaldehyde, anise alcohol, anisole, anthranilic acid methyl ester, acetophenone, benzylacetone, benzaldehyde, benzoic acid ethyl ester, benzophenone, benzyl alcohol, benzyl acetate, benzyl benzoate, benzyl formate, benzyl valerianate, bomeol, bomyl acetate, alpha-bromostyrene, n-decyl aldehyde, n-dodecyl aldehyde, eugenol, eugenol methyl ether, eucalyptol, famesol, fenchone, fenchyl acetate, geranyl acetate, geranyl formate, heliotropin, heptyne carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrole, jasmine, camphor, carvacrol, carvone, p-cresol methyl ether, cumarin, p-methoxyacetophenone, methyl n-amyl ketone, methylanthranilic acid methyl ester, p-methylacetophenone, methylchavicol, p-methylquinoline, methyl beta-naphthyl ketone, methyl n-nonylacetaldehyde, methyl n-nonyl ketone, muscone, beta-naphthol ethyl ether, beta-naphthol methyl ether, nerol, nitrobenzene, n-nonylaldehyde, nonyl alcohol, n-octylaldehyde, p-oxyacetophenone, pentadecanolide, beta-phenethyl alcohol, phenylacetaldehyde dimethyl acetal, phenylacetic acid, pulegone, safrole, salicylic acid isoamyl ester, salicylic acid methyl ester, salicylic acid hexyl ester, salicylic acid cyclohexyl ester, santalol, skatole, terpineol, thyme, thymol, gamma-undecalactone, vanillin, veratraldehyde, cinnamaldehyde, cinnamyl alcohol, cinnamic acid, cinnamic acid ethyl ester, cinnamic acid benzyl ester. The lower-volatility fragrances include in particular lower-boiling odorants of natural or synthetic origin, which may be used alone or in mixtures. Examples of lower-volatility fragrances are alkyl isothiocyanates (alkyl mustard oils), butanedione, limonene, linalool, linayl acetate and propionate, menthol, menthone, methyl-n-heptenone, phellandrene, phenylacetaldehyde, terpinyl acetate, citral, and citronellal.

According to another preferred embodiment, the agent (i.e., washing or cleaning agent, cosmetic agent, or air freshening agent) contains at least one, or multiple, active components, in particular washing, care, cleansing, and/or cosmetic components, such as anionic surfactants, cationic surfactants, amphoteric surfactants, nonionic surfactants, acidifying agents, alkalizing agents, anti-crease compounds, antibacterial substances, antioxidants, anti-redeposition agents, antistatic agents, builders, bleaching agents, bleach activators, bleach stabilizers, bleach catalysts, ironing aids, cobuilders, fragrances, shrinkage preventers, electrolytes, enzymes, color protectants, colorants, dyes, dye transfer inhibitors, fluorescing agents, fungicides, germicides, odor-complexing substances, adjuvants, hydrotropes, rinse aids, complexing agents, preservatives, corrosion inhibitors, water-miscible organic solvents, optical brighteners, perfumes, perfume carriers, luster agents, pH adjusters, proofing and impregnation agents, polymers, swelling and anti-slip agents, foam inhibitors, phyllosilicates, soil-repellent substances, silver protectants, silicone oils, soil release ingredients, UV protection substances, viscosity regulators, thickeners, discoloration inhibitors, graying inhibitors, vitamins, and/or softeners. Unless indicated otherwise, stated quantities in % by weight refer to the total weight of the agent.

The quantities of the individual ingredients in the agents (i.e., washing or cleaning agents, cosmetic agents, or air freshening agents) in each case depend on the intended purpose of the agents in question. Depending on the intended purpose of the agents, for example the surfactant content is selected to be higher or lower. The surfactant content of washing agents may typically be, for example, from about 10 to about 50% by weight, alternatively from about 12.5 to about 30% by weight, or from about 15 to about 25% by weight, while, for example, cleaning agents for automatic dishwashers may contain, for example, from about 0.1 to about 10% by weight, alternatively from about 0.5 to about 7.5% by weight, or from about 1 to about 5% by weight of surfactants.

The agents (i.e., washing or cleaning agents, cosmetic agents, or air freshening agents) may contain surfactants, such as anionic surfactants, nonionic surfactants, and the mixtures thereof, as well as cationic surfactants. Suitable nonionic surfactants may be or include, but are not limited to, ethoxylation and/or propoxylation products of alkyl glycosides and/or linear or branched alcohols in each case having from 12 to 18 carbon atoms in the alkyl portion and from 3 to 20, alternatively from 4 to 10, alkyl ether groups. Also usable are corresponding ethoxylation and/or propoxylation products of N-alkylamines, vicinal diols, fatty acid esters, and fatty acid amides which with regard to the alkyl portion correspond to the stated long-chain alcohol derivatives, and alkylphenols having from 5 to 12 carbon atoms in the alkyl functional group.

Suitable anionic surfactants may be or include, but are not limited to soaps, and those containing sulfate or sulfonate groups, such as with alkali ions as cations. Usable soaps may be the alkali salts of saturated or unsaturated fatty acids having from 12 to 18 carbon atoms. Such fatty acids may also be used in incompletely neutralized form. Usable surfactants of the sulfate type include the salts of sulfuric acid semiesters of fatty alcohols having from 12 to 18 carbon atoms, and the sulfation products of the stated nonionic surfactants with a low ethoxylation number. The usable surfactants of the sulfonate type include linear alkylbenzenesulfonates having from 9 to 14 carbon atoms in the alkyl portion, alkanesulfonates having from 12 to 18 carbon atoms, and olefin sulfonates having from 12 to 18 carbon atoms, resulting from the reaction of corresponding monoolefins with sulfur trioxide, and alpha-sulfo fatty acid esters resulting from the sulfonation of fatty acid methyl or ethyl esters.

Cationic surfactants may be or include, but are not limited to, esterquats and/or quaternary ammonium compounds (QACs) according to the general formula (R^{I})(R^{II})(R^{III})(R^{IV})N⁺X⁻, in which R^{I} through R^{IV} stand for C₁₋₂₂ alkyl functional groups, C₇₋₂₈ arylalkyl functional groups, or heterocyclic functional groups, which are the same or different, where two functional groups, or, in the case of an aromatic bond such as in pyridine, even three functional groups, together with the nitrogen atom form the heterocycle, for example a pyridinium or imidazolinium compound, and X⁻ stands for halide ions, sulfate ions, hydroxide ions, or similar anions. QACs may be prepared by reacting tertiary amines with alkalizing agents, for example methyl chloride, benzyl chloride, dimethyl sulfate, dodecyl bromide, or also ethylene oxide. The alkylation of tertiary amines with a long alkyl functional group and two methyl groups is particularly simple; in addition, the quaternization of tertiary amines with two long functional groups and a methyl group may be carried out under mild conditions, using methyl chloride. Amines having three long alkyl functional groups or hydroxy-substituted alkyl functional groups are less reactive, and are quaternized with dimethyl sulfate, for example. Examples of suitable QACs are benzalkonium chloride (N-alkyl-N,N-dimethylbenzylammonium chloride), Benzalkon B (m,p-dichlorobenzyldimethyl-C₁₂ alkylammonium chloride, benzoxonium chloride (benzyldodecyl-bis-(2-hydroxyethyl) ammonium chloride), cetrimonium bromide (N-hexadecyl-N,N-trimethylammonium bromide), benzethonium chloride (N,N-dimethyl-N-[2-[2-[p-(1,1,3,3-tetramethylbutyl)phenoxy]ethoxy]ethyl]benzylammonium chloride), dialkyldimethylammonium chlorides such as di-n-decyldimethyl ammonium chloride, didecyldimethyl ammonium bromide, dioctyldimethyl ammonium chloride, 1-cetylpyridinium chloride, and thiazoline iodide, and the mixtures thereof. Preferred QACs are the benzalkonium chlorides having C₈-C₂₂ alkyl functional groups, in particular C₁₂-C₁₄ alkyl benzyldimethylammonium chloride.

The esterquats may be or include, but are not limited to, methyl-N-(2-hydroxyethyl)-N,N-di(talgacyloxyethyl) ammonium methosulfate, bis-(palmitoyl)ethylhydroxyethylmethyl ammonium methosulfate, or methyl-N,N-bis(acyloxyethyl)-N-(2-hydroxyethyl) ammonium methosulfate. Examples of commercially available products are the methylhydroxyalkyldialkoyloxyalkyl ammonium methosulfates marketed by Stepan under the trademark Stepantex®, the products from BASF SE known under the trade name Dehyquart, or the products from Evonik known under the name Rewoquat.

Surfactants may be contained in the agents (i.e., washing or cleaning agents, cosmetic agents, or air freshening agents) in an amount ranging from about 5% by weight to about 50% by weight, alternatively from about 8% by weight to about 30% by weight, or up to 30% by weight in another non-limiting embodiment, such as from about 5% by weight to about 15% by weight, of surfactants, and among these, cationic surfactants at least in part, may be used in particular in laundry aftertreatment agents.

An agent, in particular a washing or cleaning agent, may include at least one water-soluble and/or water-insoluble, organic and/or inorganic builder. The water-soluble organic builders include polycarboxylic acids, in particular citric acid and sugar acids, monomeric and polymeric aminopolycarboxylic acids, in particular methylglycinediacetic acid, nitrilotriacetic acid, and ethylenediaminetetraacetic acid as well as polyaspartic acid, polyphosphonic acids, in particular amino tris(methylenephosphonic acid), ethylenediaminetetrakis(methylenephosphonic acid), and 1-hydroxyethane-1,1-diphosphonic acid, polymeric hydroxy compounds such as dextrin, and polymeric (poly)carboxylic acids, polymeric acrylic acids, methacrylic acids, maleic acids, and mixed polymers thereof, which may also contain small portions of polymerizable substances, without a carboxylic acid functionality, in the polymer. Compounds of this class which are suitable, may be copolymers of acrylic acid or methacrylic acid with vinyl ethers, such as vinyl methyl ethers, vinyl esters, ethylene, propylene, and styrene, in which the proportion of the acid is at least 50% by weight. The organic builders, in particular for production of liquid agents, may be used in the form of aqueous solutions, such as in the form of 30 to 50% by weight aqueous solutions. All stated acids are generally used in the form of their water-soluble salts, in particular their alkali salts.

Organic builders, if desired, may be included in the agent in an amount up to 40% by weight, alternatively up to 25% by weight, or from about 1% by weight to about 8% by weight. Quantities near the stated upper limit may be used in paste-form or liquid, such as water-containing, agents. Laundry aftertreatment agents, such as softeners, may optionally also be free of organic builders.

In particular alkali silicates and polyphosphates, e.g. sodium triphosphate, are suitable as water-soluble inorganic builder materials. In particular crystalline or amorphous alkali aluminosilicates, if desired, may be used in quantities of up to 50% by weight, alternatively not above 40% by weight, and in liquid agents, in an amount ranging from about 1% by weight to about 5% by weight, as water-insoluble, water-dispersible inorganic builder materials. Among these, the crystalline sodium aluminosilicates in washing agent quality, in particular zeolite A, P, and optionally X, may be used as inorganic builder materials. Quantities near the stated upper limit may be used in solid particulate agents. Suitable aluminosilicates may have no particles with a grain size greater than 30 μm, such as at least 80% by weight of particles with a size smaller than 10 μm.

Suitable substitutes or partial substitutes for the stated aluminosilicate are crystalline alkali silicates, which may be present alone or in a mixture with amorphous silicates. The alkali silicates that are usable in the agents as builders have a molar ratio of alkali oxide to SiO₂ of less than 0.95, such as 1:1.1 to 1:12, and may be present in amorphous or crystalline form. Alkali silicates may include sodium silicates, such as amorphous sodium silicates having a Na₂O:SiO₂ molar ratio of 1:2 to 1:2.8. Other non-limiting examples of crystalline silicates, which may be present alone or in a mixture with amorphous silicates, are crystalline phyllosilicates of the general formula Na₂SiₓO₂ₓ₊₁.y H₂O, in which x, the so-called module, is a number from 1.9 to 4 and y is a number from 0 to 20, or where the values for x are 2, 3, or 4. Non-limiting crystalline phyllosilicates are those in which x in the stated general formula assumes the values 2 or 3. In particular, beta- as well as delta-sodium disilicates (Na₂Si₂O₅.y H₂O) may also be used. Crystalline alkali silicates of the above general formula, in which x is a number from 1.9 to 2.1, which are prepared from amorphous alkali silicates and are practically water-free, may be used in the agents. In another embodiment of agents, a crystalline sodium phyllosilicate having a module of 2 to 3, which may be prepared from sand and soda, is used. Crystalline sodium silicates having a module in the range of 1.9 to 3.5 are used in another embodiment of agents. If alkali aluminosilicate, in particular zeolite, is also present as additional builder, the weight ratio of aluminosilicate to silicate, in each case based on water-free active substances, ranges from 1:10 to 10:1. In agents containing amorphous as well as crystalline alkali silicates, the weight ratio of amorphous alkali silicate to crystalline alkali silicate may be from 1:2 to 2:1 and in particular 1:1 to 2:1.

Builders, if desired, are contained in the agents in quantities of up to 60% by weight, such as from about 5% by weight to about 40% by weight. Laundry aftertreatment agents, for example softeners, may be free of inorganic builders.

In particular organic peracids or peracid salts of organic acids, such as phthalimidopercapronic acid, perbenzoic acid, or salts of diperdodecandioic acid, hydrogen peroxide, and inorganic salts that release hydrogen peroxide under the use conditions, such as perborate, percarbonate, and/or persilicate, are suitable as peroxygen compounds. If solid peroxygen compounds are to be employed, these may be used in the form of powders or granules, which in principle may also be coated in a known manner. The optional use of alkali percarbonate, alkali perborate monohydrate, or in particular in liquid agents, hydrogen peroxide in the form of aqueous solutions containing from about 3% by weight to about 10% by weight hydrogen peroxide may be used. If an agent contains bleaching agents, such as peroxygen compounds, these may be present in quantities of up to 50% by weight, such as from about 5% by weight to about 30% by weight. The addition of small quantities of known bleaching agent stabilizers such as phosphonates, borates or metaborates, and metasilicates, as well as magnesium salts such as magnesium sulfate, may be used.

Compounds, which under perhydrolysis conditions, result in aliphatic peroxocarboxylic acids having from 1 to 10 carbon atoms, such as from 2 to 4 carbon atoms, and/or optionally substituted perbenzoic acid, may be used as bleach activators. Substances that bear the 0- and/or N-acyl groups of the stated C atomic number and/or optionally substituted benzoyl groups are suitable. Non-limiting examples of the aliphatic peroxocarboxylic acids may include may be acylated alkylene diamines, such as tetraacetylethylenediamine (TAED), acylated triazine derivatives, such as 1,5-diacetyl-2,4-dioxohexahydro-1,3,5-triazine (DADHT), acylated glycolurils, such as tetraacetylglycoluril (TAGU), N-acylimides, such as N-nonanoyl succinimide (NOSI), acylated phenolsulfonates, such as n-nonanoyl- or isononanoyloxybenzenesulfonate (n- or iso-NOBS), carboxylic acid anhydrides, such as phthalic acid anhydride, acylated polyhydric alcohols, such as triacetin, ethylene glycol diacetate, 2,5-diacetoxy-2,5-dihydrofuran, and enol esters, as well as acetylated sorbitol and mannitol or the mixtures thereof (SORMAN), acylated sugar derivatives, such as pentaacetyl glucose (PAG), pentaacetyl fructose, tetraacetyl xylose, and octaacetyl lactose, as well as acetylated, optionally N-alkylated glucamine and gluconolactone, and/or N-acylated lactams, for example N-benzoylcaprolactam. Hydrophilically substituted acyl acetals and acyl lactams may be used. Combinations of conventional bleach activators may also be used. These types of bleach activators may be contained in a customary quantity range, such in quantities of 1% by weight to 10% by weight, alternatively 2% by weight to 8% by weight, based on the overall agent.

In addition to or instead of the conventional bleach activators listed above, sulfonimines and/or bleach-enhancing transition metal salts or transition metal complexes may be contained as so-called bleach catalysts.

Suitable as enzymes that are usable in the agents are those from the class of proteases, cutinases, amylases, pullulanases, hemicellulases, cellulases, lipases, oxidases, and peroxidases, and the mixtures thereof. Enzymatic active ingredients obtained from fungi or bacteria, such as Bacillus subtilis, Bacillus licheniformis, Streptomyces griseus, Humicola lanuginosa, Humicola insolens, Pseudomonas pseudoalcaligenes, or Pseudomonas cepacia may be suited. The optionally used enzymes may be adsorbed onto carrier substances and/or embedded in coating substances to protect the enzymes from premature inactivation. The enzymes, if desired, may be contained in the agents according to the invention in quantities no greater than 5% by weight, in particular 0.2% by weight to 2% by weight.

The agents may optionally contain, for example, derivatives of diaminostilbene disulfonic acid or the alkali metal salts thereof as optical brighteners. Suitable, for example, are salts of 4,4′-bis(2-anilino-4-morpholino-1,3,5-triazinyl-6-amino)stilbene-2,2′-disulfonic acid or compounds having a similar structure, which instead of the morpholino group bear a diethanolamino group, a methylamino group, an anilino group, or a 2-methoxyethylamino group.

Suitable foam inhibitors include, for example, organopolysiloxanes and the mixtures thereof with microfine, optionally silanated silicic acid and paraffin waxes and the mixtures thereof with silanated silicic acid or bis-fatty acid alkylene diamides. Mixtures of various foam inhibitors are also used, for example those made up of silicones, paraffins, or waxes. The foam inhibitors, in particular silicone- and/or paraffin-containing foam inhibitors, may be bound to a granular carrier substance that is soluble or dispersible in water. Mixtures of paraffin waxes and bistearylethylenediamides may be used.

Furthermore, the agents may also contain components that positively influence the capability for washing out oil and grease from textiles, so-called soil release active ingredients. This effect is particularly apparent when a textile is soiled which has been previously washed several times with an agent according to the invention that contains this oil- and grease-dissolving component. Non-limiting examples of oil- and grease-dissolving components include nonionic cellulose ethers such as methylcellulose and methylhydroxypropylcellulose with a proportion of 15 to 30% by weight of methoxyl groups and 1 to 15% by weight of hydroxypropoxyl groups, in each case based on the nonionic cellulose ether, and the polymers of phthalic acid and/or terephthalic acid, known from the prior art, or the derivatives thereof with monomeric and/or polymeric diols, in particular polymers of ethylene terephthalates and/or polyethylene glycol terephthalates or anionically and/or nonionically modified derivatives thereof.

The agents may also contain dye transfer inhibitors, in quantities of 0.1% by weight to 2% by weight, in particular 0.1% by weight to 1% by weight, which in one embodiment are polymers of vinylpyrrolidone, vinyl imidazole, vinyl pyridine-N-oxide, or copolymers thereof.

The function of graying inhibitors is to keep the dirt that is removed from the textile fiber suspended in the liquor. Water-soluble colloids, which are usually organic, are suitable for this purpose, for example starch, sizing material, gelatin, salts of ethercarboxylic acids or ethersulfonic acids of starch or of cellulose, or salts of acidic sulfuric acid esters of cellulose or of starch. Water-soluble polyamides containing acidic groups are also suitable for this purpose. Starch derivatives other than those mentioned above may also be used, for example aldehyde starches. Cellulose ethers such as carboxymethylcellulose (Na salt), methylcellulose, hydroxyalkylcellulose, and mixed ethers such as methylhydroxyethylcellulose, methylhydroxypropylcellulose, methylcarboxymethylcellulose, and the mixtures thereof may preferably be used, for example in quantities of 0.1 to 5% by weight, based on the agents.

The organic solvents that are usable in the agents, in particular when the agents are present in liquid or paste-like form, include alcohols having 1 to 4 carbon atoms, such as methanol, ethanol, isopropanol, and tert-butanol, diols having from 2 to 4 carbon atoms, in particular ethylene glycol and propylene glycol, and the mixtures thereof, and the ethers that are derivable from the mentioned compound classes. Such water-miscible solvents are present in the agents according to the invention in quantities of not greater than 30% by weight, e.g. 6% by weight to 20% by weight.

For setting a desired pH that does not result from mixing the other components themselves, the agents may contain acids that are compatible with the system and the environment, in particular citric acid, acetic acid, tartaric acid, malic acid, lactic acid, glycolic acid, succinic acid, glutaric acid, and/or adipic acid, as well as mineral acids, in particular sulfuric acid, or bases, in particular ammonium or alkali hydroxides. These types of pH regulators are optionally contained in the agents according to the invention, preferably in quantities no greater than 20% by weight, in particular 1.2% by weight to 17% by weight.

The production of solid agents (i.e., washing or cleaning agents) poses no difficulties, and in principle may take place in a known manner, for example by spray drying or granulation, with an optional peroxygen compound and an optional bleach catalyst being optionally added later. For production of agents having an increased bulk weight, in particular in the range of 650 g/L to 950 g/L, a method having an extrusion step may be used. The production of liquid agents also poses no difficulties, and may likewise take place in a known manner.

Preparation of the compounds of formula (I) is described in the Examples section by way of example, with reference to the preparation of a pro-fragrance containing citronellol or geraniol. The other compounds of general formula (I), and in particular the compounds of formulas (II) through (XIII), may also be prepared via this basic synthesis route.

According to one embodiment, the teaching may be used to significantly reduce the perfume portion in the washing, cleaning, and body care agents. It is thus possible to also offer perfumed products to particularly sensitive consumers who, due to specific intolerances and irritations, can use the normally perfumed products only on a limited basis or not at all.

In various embodiments of the present invention, the washing or cleaning agents are present in liquid or in solid form.

A solid, e.g. powdered, washing agent, in addition to the compound, may also contain additional components, for example:
anionic surfactants, such as alkylbenzenesulfonate, alkyl sulfate, for example in quantities from 5 to 30% by weight,
nonionic surfactants, such as fatty alcohol polyglycol ether, alkyl polyglucoside, fatty acid glucamide, for example in quantities from 0.5 to 15% by weight,
builders, for example zeolite, polycarboxylate, sodium citrate, in quantities of for example 0 to 70% by weight, or from 5 to 60% by weight, or from 10 to 55% by weight, or from 15 to 40% by weight,
alkalis, for example sodium carbonate, in quantities of for example 0 to 35% by weight, or from 1 to 30% by weight, or from 2 to 25% by weight, or from 5 to 20% by weight,
bleaching agents, for example sodium perborate, sodium percarbonate, in quantities of for example 0 to 30% by weight, or from 5 to 25% by weight, or from 10 to 20% by weight,
corrosion inhibitors, for example sodium silicate, in quantities of for example 0 to 10% by weight, or from 1 to 6% by weight, or from 2 to 5% by weight, or from 3 to 4% by weight,
stabilizers, for example phosphonates, from 0 to 1% by weight,
foam inhibitors, for example soap, silicone oils, paraffins, from 0 to 4% by weight, or from 0.1 to 3% by weight, or from 0.2 to 1% by weight,
enzymes, for example proteases, amylases, cellulases, lipases, from 0 to 2% by weight, or from 0.2 to 1% by weight, or from 0.3 to 0.8% by weight,
graying inhibitors, for example carboxymethylcellulose, from 0 to 1% by weight,
discoloration inhibitors, for example polyvinylpyrrolidone derivatives, from 0 to 2% by weight,
adjusters, for example sodium sulfate, from 0 to 20% by weight,
optical brighteners, for example stilbene derivatives, biphenyl derivatives, from 0 to 0.4% by weight, or from 0.1 to 0.3% by weight,
optionally further fragrances,
optionally water,
optionally soap,
optionally bleach activators,
optionally cellulose derivatives,
optionally soil repellent agents, in % by weight, in each case based on the overall agent.

In another preferred embodiment of the invention, the agent is present in liquid form, preferably in gel form. Preferred liquid washing or cleaning agents and cosmetics have water contents of for example 10 to 95% by weight, or from 20 to 80% by weight, and or from 30 to 70% by weight, based on the overall agent. In the case of liquid concentrates, the water content may also be particularly low, for example <30% by weight, or <20% by weight, or <15% by weight, in % by weight in each case based on the overall agent. The liquid agents may also contain nonaqueous solvents.

A non-limiting liquid, such as gel-form, washing agent, in addition to the compound, may also contain in particular components that are selected from the following, for example:
anionic surfactants, such as preferably alkylbenzenesulfonate, alkyl sulfate, for example in quantities of 5 to 40% by weight,
nonionic surfactants, such as preferably fatty alcohol polyglycol ether, alkyl polyglucoside, fatty acid glucamide, for example in quantities of 0.5 to 25% by weight,
builders, for example zeolite, polycarboxylate, sodium citrate, 0 to 15% by weight, or 0.01 to 10% by weight, or from 0.1 to 5% by weight,
foam inhibitors, for example soap, silicone oils, paraffins, in quantities of for example 0 to 10% by weight, or from 0.1 to 4% by weight, or from 0.2 to 2% by weight, or from 1 to 3% by weight,
enzymes, for example proteases, amylases, cellulases, lipases, in quantities of for example 0 to 3% by weight, or from 0.1 to 2% by weight, or from 0.2 to 1% by weight, or from 0.3 to 0.8% by weight,
optical brighteners, for example stilbene derivatives, biphenyl derivatives, in quantities of for example 0 to 1% by weight, or from 0.1 to 0.3% by weight, or from 0.1 to 0.4% by weight,
optionally further fragrances,
optionally stabilizers,
water,
optionally soap, in quantities of for example 0 to 25% by weight, or from 1 to 20% by weight, or from 2 to 15% by weight, or from 5 to 10% by weight,
optionally solvents (preferably alcohols), 0 to 25% by weight, or from 1 to 20% by weight, or from 2 to 15% by weight, in % by weight, in each case based on the overall agent.

A non-limiting liquid softener, in addition to the ketone, may also contain components, such as:
cationic surfactants, such as in particular esterquats, for example in quantities of 5 to 30% by weight,
cosurfactants, for example glycerol monostearate, stearic acid, fatty alcohols, fatty alcohol ethoxylates, for example in quantities of 0 to 5% by weight, or from 0.1 to 4% by weight,
emulsifiers, for example fatty amine ethoxylates, for example in quantities of 0 to 4% by weight, or from 0.1 to 3% by weight,
optionally further fragrances,
dyes, preferably in the ppm range,
stabilizers, preferably in the ppm range,
solvents, for example water, in quantities of from 60 to 90% by weight, in % by weight, in each case based on the overall agent.

A further subject matter of the invention relates to a method for the long-lasting scenting of surfaces, wherein the compound having formula (I) or the washing or cleaning agent, cosmetic agent, or air freshening agent is applied to the surface to be scented (textiles, dishes, floors, for example), and this surface is subsequently exposed to electromagnetic radiation having wavelengths ranging from about 200 to about 600 nm.

### EXAMPLES

### Example 1: Synthesis of Citral Derivatives

Geranioxy Acetonitrile

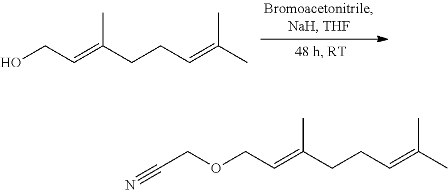

Geraniol (10 g, 64.83 mmol) was slowly added dropwise to a suspension of sodium hydride (3.11 g, 77.8 mmol, 60% in paraffin oil) in tetrahydrofuran (100 mL). The reaction solution was stirred under reflux for 2.5 h and subsequently cooled to room temperature. Bromoacetonitrile (5.43 mL, 77.8 mmol) was slowly added dropwise over a period of 1.5 h, with ice cooling. The dark brown reaction solution was then stirred for 48 h at room temperature. The reaction was terminated by adding saturated aqueous ammonium chloride solution. The phases were separated, the aqueous phase was extracted with diethyl ether (3×100 mL), the combined organic phases were washed with saturated aqueous sodium chloride solution, dried with sodium sulfate, and filtered, and the solvent was removed under reduced pressure. The crude product was purified by column chromatography (PE:EE 10:1), and the geranioxy acetonitrile (6.37 g, 32.96 mmol, 51%) was obtained.

4-Methylphenacyl Geraniol

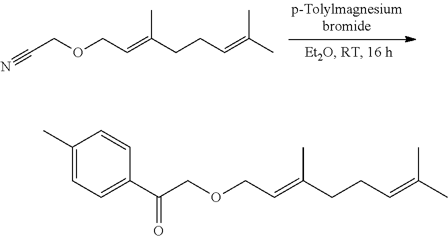

Geranioxy acetonitrile (3.70 g, 19.14 mmol) was dissolved in diethyl ether (100 mL). p-Tolylmagnesium bromide (46 mL, 22.97 mmol, 0.5 M in diethyl ether) was then added dropwise over a period of 10 minutes, with ice cooling, and the reaction solution was stirred for 16 h at room temperature. The reaction was terminated by adding 5% HCl (200 mL), and the mixture was heated under reflux for 2.5 h. The mixture was then cooled to room temperature, the phases were separated, and the aqueous phase was extracted with dichloromethane (3×100 mL). The combined organic phases were dried with magnesium sulfate and filtered, and the solvent was removed under reduced pressure. The crude product was purified by column chromatography, and 4-methylphenacyl geraniol (4.0 g, 14.0 mmol, 73%) was obtained.

Phenacyl Geraniol

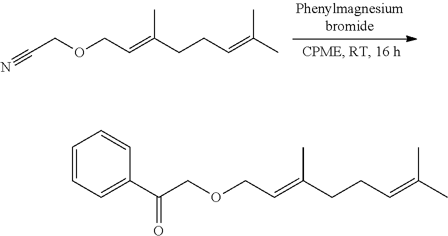 [SMILES: CC(C)=CCC/C(C)=C/COCC#N ; CC(C)=CCC/C(C)=C/COCC(=O)c1ccccc1]

Geranioxy acetonitrile (3.7 g, 19.14 mmol) was dissolved in cyclopentyl methyl ether (100 mL). Phenylmagnesium bromide (15 mL, 22.97 mmol, 1.6 M in cyclopentyl methyl ether) was then added dropwise over a period of 10 minutes, with ice cooling, and the reaction solution was stirred for 16 h at room temperature. The reaction was terminated by adding 5% HCl (200 mL), and the mixture was heated under reflux for 2.5 h. The mixture was then cooled to room temperature, the phases were separated, and the aqueous phase was extracted with dichloromethane (3×100 mL). The combined organic phases were dried with magnesium sulfate and filtered, and the solvent was removed under reduced pressure. The crude product was purified by column chromatography, and phenacyl geraniol (3.65 g, 13.4 mmol, 69%) was obtained.

### Example 2: Synthesis of Citronellal Derivatives

Citronelloxy Acetonitrile

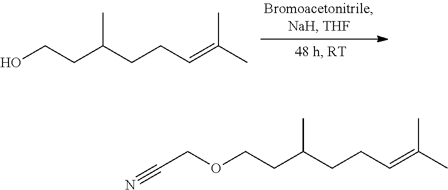

Citronellol (10 g, 63.99 mmol) was slowly added dropwise to a suspension of sodium hydride (3.07 g, 76.79 mmol, 60% in paraffin oil) in tetrahydrofuran (100 mL). The reaction solution was stirred under reflux for 2.5 h and subsequently cooled to room temperature. Bromoacetonitrile (5.35 mL, 76.79 mmol) was slowly added dropwise over a period of 1.5 h, with ice cooling. The dark brown reaction solution was then stirred for 48 h at room temperature. The reaction was terminated by adding saturated aqueous ammonium chloride solution. The phases were separated, the aqueous phase was extracted with diethyl ether (3×100 mL), the combined organic phases were washed with saturated aqueous sodium chloride solution, dried with sodium sulfate, and filtered, and the solvent was removed under reduced pressure. The crude product was purified by column chromatography (PE:EE 10:1), and citronelloxy acetonitrile (3.29 g, 16.85 mmol, 26%) was obtained.

4-Methylphenacyl Citronellol

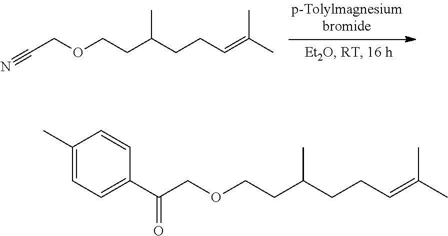

Citronelloxy acetonitrile (2.81 g, 14.39 mmol) was dissolved in diethyl ether (100 mL). p-Tolylmagnesium bromide (34.54 mL, 17.27 mmol, 0.5 M in diethyl ether) was then added dropwise over a period of 10 minutes, with ice cooling, and the reaction solution was stirred for 16 h at room temperature. The reaction was terminated by adding 5% HCl (200 mL), and the mixture was heated under reflux for 2.5 h. The mixture was then cooled to room temperature, the phases were separated, and the aqueous phase was extracted with dichloromethane (3×100 mL). The combined organic phases were dried with magnesium sulfate and filtered, and the solvent was removed under reduced pressure. The crude product was purified by column chromatography, and 4-methylphenyl citronellol (2.22 g, 7.70 mmol, 53%) was obtained.

Phenacyl Citronellol

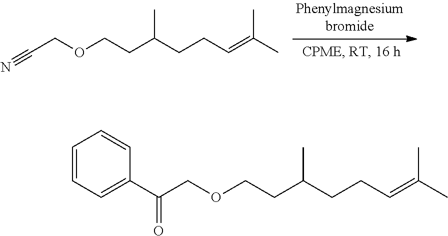 [SMILES: CC(C)=CCCC(C)CCOCC#N ; CC(C)=CCCC(C)CCOCC(=O)c1ccccc1]

Citronelloxy acetonitrile (3.29 g, 16.85 mmol) was dissolved in cyclopentyl methyl ether (100 mL). Phenylmagnesium bromide (12.63 mL, 20.21 mmol, 1.6 M in cyclopentyl methyl ether) was then added dropwise over a period of 10 minutes, with ice cooling, and the reaction solution was stirred for 16 h at room temperature. The reaction was terminated by adding 5% HCl (200 mL), and the mixture was heated under reflux for 2.5 h. The mixture was then cooled to room temperature, the phases were separated, and the aqueous phase was extracted with dichloromethane (3×100 mL). The combined organic phases were dried with magnesium sulfate and filtered, and the solvent was removed under reduced pressure. The crude product was purified by column chromatography, and phenacyl citronellol (3.03 g, 11.0 mmol, 66%) was obtained.

### Example 3: Yellowing

Yellowing

| Compound | Yes | No |
|---|---|---|
| Reference compound | x | |
| 4-Methoxyphenacyl citronellol | | |
| (reference/comparison compound) KRO 62 | | |
| 4-Methylphenacyl geraniol KRO 60 | | x |
| Phenacyl citronellol KRO 58 | | x |
| 4-Methylphenacyl citronellol KRO 60 | | x |
| Phenacyl citronellol | | x |

Reference Compound KRO 62

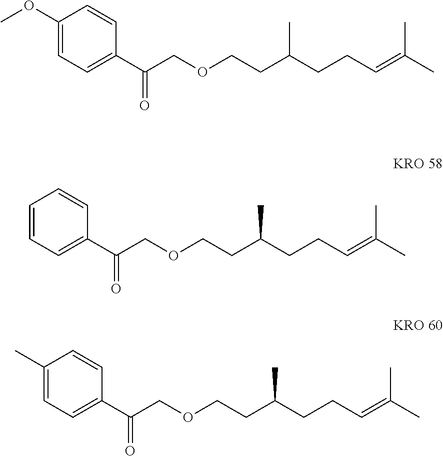

In addition, measurements by fluorescence spectroscopy have shown that for compound KRO 62, excitation is most intense at 325 nm, which, however, is disadvantageous for the fragrance release, since intense fluorescence means that the fragrance is not released. Thus, the compounds KRO 58 and KRO 60 are also advantageous with regard to the desired fragrance release, due to the fact that the excitation with light of wavelength 325 nm results in splitting of the bond and release of the fragrance.

### Example 4: Fragrance Release Performance

The fabric swatches washed with Vemel conc. base composition with the test substances added were irradiated at 0.58 W/cm² for 1 min, using an Atlas Suntest XXL+ sunlight simulator. The fabric swatches were situated in a Petri dish with a quartz glass cover (transparent to the entire light spectrum). Before and after the irradiation, the intensity of the fragrance was assessed by smell and rated on a scale of 1 to 10 (10=intense odor, 0=no odor). The intensity of release of the novel synthesized substances 4-methylphenacyl geraniol and phenacyl geraniol as well as 4-methylphenacyl citronellol and phenacyl citronellol was compared to the fragrances citral and citronellal.

TABLE 4

| | Odorant | | Intensity | Intensity |
|---|---|---|---|---|
| | content | Dosage | before | after |
| Substance | [%] | [%] | irradiation | irradiation |
| Citronellal | 100 | 0.4 | 2 | 1 |
| Citral | 100 | 0.4 | 2 | 3 |
| 4-Methylphenacyl geraniol | 53.2 | 0.76 | 2 | 8 |
| 4-Methylphenacyl citronellol | 53.8 | 0.75 | 2 | 7 |
| Phenacyl citronellol | 56.6 | 0.71 | 4-5 | 6-7 |
| 4-Methylphenacylmethyl | 51 | 0.78 | 5 | 8 |
| geraniol (formula (V)) | | | | |

The splitting of the fragrance under the action of light is shown in the following diagram, using 4-methylphenacyl geraniol and phenacyl geraniol as examples, in which citral and 1-(4-methylphenyl)ethanone and 1-phenylethanone, respectively, result as cleavage products:

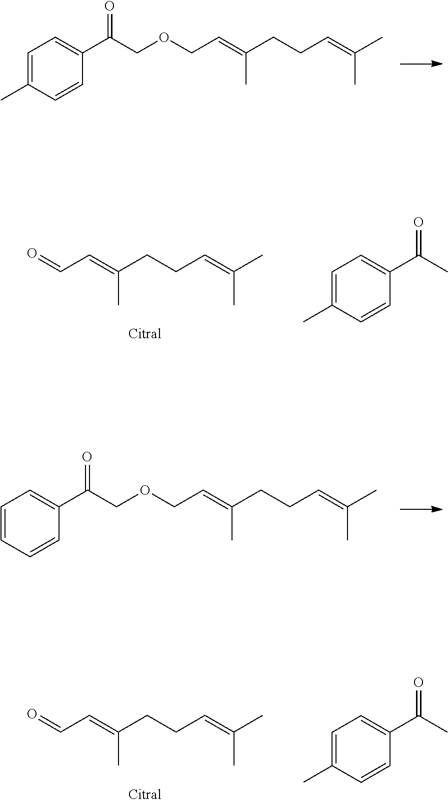

1. A compound of formula (I), 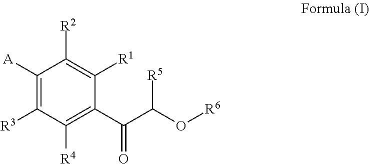
where
A is selected from the group consisting of hydrogen; substituted or unsubstituted linear or branched alkyl, alkenyl, or alkynyl having up to 20; carbon atoms, substituted or unsubstituted linear or branched heteroalkyl, heteroalkenyl, or heteroalkynyl having up to 20 carbon atoms, and from 1 to 6 heteroatoms selected from the group consisting of O, S, and N; substituted or unsubstituted aryl having up to 20 carbon atoms; substituted or unsubstituted heteroaryl having up to 20 carbon atoms, and from 1 to 6 heteroatoms selected from the group consisting of O, S, and N; cycloalkyl or cycloalkenyl having up to 20, preferably p to 12, carbon atoms; and heterocycloalkyl or heterocycloalkenyl having up to 20 carbon atoms, and from 1 to 6 heteroatoms selected from the group consisting of O, S, and N;
R¹, R², R³, R⁴, and R⁵ are independently selected from the group consisting of hydrogen; —OHM; —NH₂; halogen; substituted or unsubstituted linear or branched alkyl, alkenyl, or alkynyl having up to 20 carbon atoms; substituted or unsubstituted linear or branched heteroalkyl, heteroalkenyl, or heteroalkynyl having up to 20 carbon atoms, and from 1 to 6 heteroatoms selected from the group consisting of O, S, and N; substituted or unsubstituted aryl having up to 20 carbon atoms; substituted or unsubstituted heteroaryl having up to 20 carbon atoms, and from 1 to 6 heteroatoms selected from the group consisting of O, S, and N cycloalkyl or cycloalkenyl having up to 20 carbon atoms; and heterocycloalkyl or heterocycloalkenyl having up to 20 carbon atoms, and from 1 to 6 heteroatoms selected from the group consisting of O, S, and N; and
R⁶ is a linear, branched, or cyclic, substituted or unsubstituted hydrocarbon functional group having from 6 to 30 carbon atoms and from 0 to 10 heteroatoms selected from the group consisting of N, O, S, and Si.

2. The compound of claim 1, wherein R¹, R⁴, and R⁵, independently of one another, are selected from the functional group consisting of a hydrogen or a methyl.

3. The compound of claim 1, wherein R² and R³, independently of one another, are selected from the functional group consisting of a hydrogen or a substituted or unsubstituted linear or branched alkyl having from 1 to 5 carbon atoms.

4. The compound claim 1, wherein A is selected from the functional group consisting of a hydrogen or a linear alkyl having from 1 to 3 carbon atoms.

5. The compound claim 1, wherein R⁶ is selected from the group consisting of a substituted or unsubstituted linear or branched alkyl having from 6 to 20 carbon atoms; a substituted or unsubstituted linear or branched alkenyl having from 6 to 20 carbon atoms; a substituted or unsubstituted alkylaryl having from 6 to 20 carbon atoms; a substituted or unsubstituted aryl having from 6 to 20 carbon atoms; a cycloalkyl having from 6 to 20 carbon atoms; and a cycloalkenyl having from 6 to 20 carbon atoms.

6. The compound claim 1, wherein R⁶ is a functional group that is derived from a fragrance aldehyde or a fragrance ketone, wherein the fragrance ketone or the fragrance aldehyde is selected from the group consisting of geranial, neral, (R)-citronellal, (S)-citronellal, buccoxime, isojasmone, methyl beta-naphthyl ketone, musk indanone, Tonalid/Musk Plus, alpha-damascone, beta-damascone, delta-damascone, gamma-damascone, damascenone, damarose, methyl dihydrojasmonate, menthone, carvone, hedione, camphor, fenchone, alpha-ionone, beta-ionone, gamma-methylionone, fleuramone, dihydrojasmone, cis-jasmone, Iso E Super, methyl cedrenyl ketone, methyl cedrylone, acetophenone, methylacetophenone, para-methoxyacetophenone, benzylacetone, benzophenone, para-hydroxyphenylbutanone, celery ketone, livescone, 6-isopropyldecahydro-2-naphtone, dimethyl octenone, Frescomenthe, 4-(1-ethoxyvinyl)-3,3,5,5-tetramethylcyclohexanone, methyl heptenone, 2-(2-(4-methyl-3-cyclohexen-1-yl)propyl)cyclopentanone, 1-(p-menthen-6(2)yl)-1-propanone, 4-(4-hydroxy-3-methoxyphenyl)-2-butanone, 2-acetyl-3,3-dimethylnorbornane, 6,7-dihydro-1,1,2,3,3-pentamethyl-4(5h)indanone, 4-damascol, dulcinyl, Cassione, gelsone, hexalone, Isocyclemone E, methyl cyclocitrone, methyl lavender ketone, orivone, para-tertiary butylcyclohexanone, verdone, Delphone, muscone, neobutenone, Plicatone, Veloutone, 2,4,4,7-tetramethyloct-6-en-3-one, and tetramerane.

7. The compound claim 1, wherein the compound is selected from one of the following formulas (II) through (XXV):

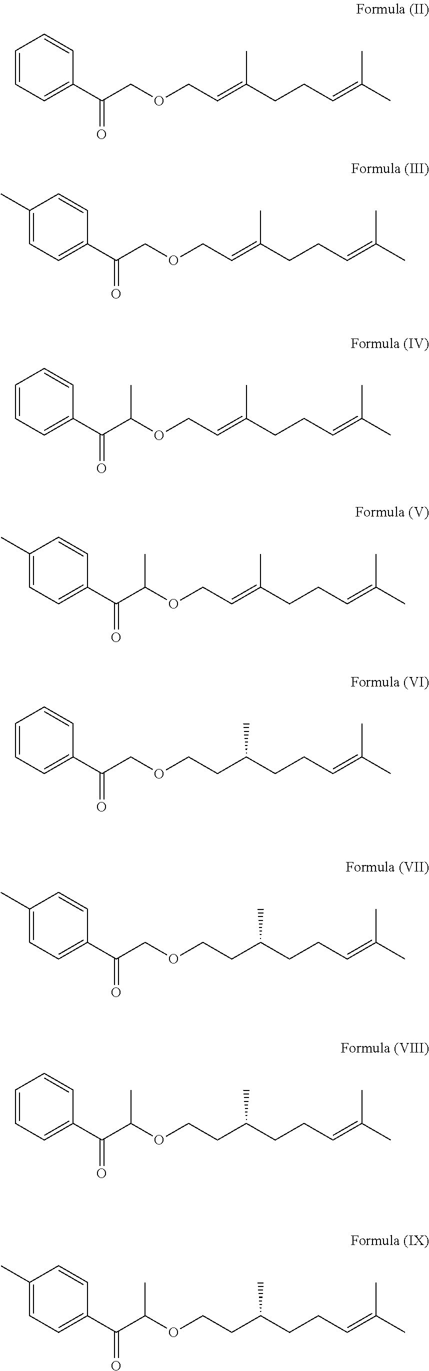

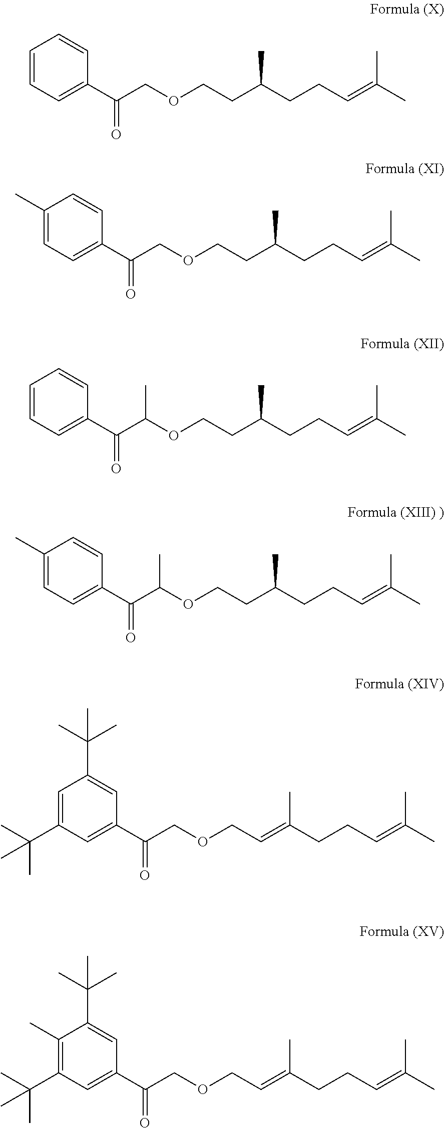

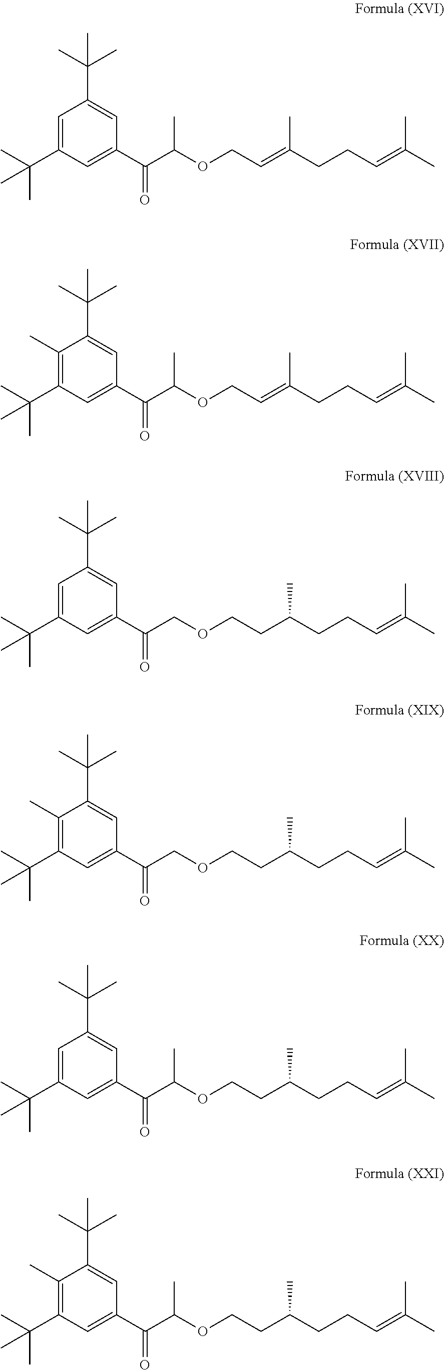

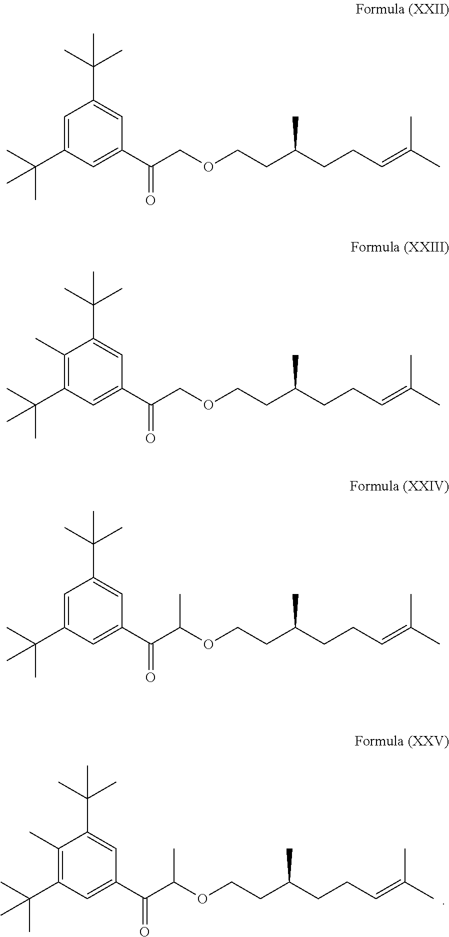

8. A composition comprising the compound of claim 1, wherein the composition is selected from the group consisting of a washing agent, a cleaning agent, an air freshening agent, a cosmetic agent, and combinations thereof.

9. The compound of claim 8, wherein the compound is a washing agent or a cleaning agent; and wherein the compound comprises at least one of:
a. an amount of the at least one compound ranges from about 0.0001 to about 5% by weight; based on the overall washing or cleaning agent
b. at least one surfactant selected from the group consisting of an anionic surfactant, a cationic surfactant, a nonionic surfactant, a zwitterionic surfactant, an amphoteric surfactant, and mixtures thereof;
c. the compound is present in liquid or solid form; and
d. combinations thereof.

10. The compound of claim 8, wherein the air freshening agent comprises the compound in, an amount ranging from about 0.0001 to about 50% by weight based on the overall air freshening agent.

11. The compound of claim 8, wherein the cosmetic agent comprises the compound in, an amount ranging from about 0.0001 to about 50% by weight based on the overall cosmetic agent.

12. A method for the long-lasting scenting of surfaces comprising:
applying at least one compound from claim 1 to at least one surface to be scented; and
exposing the at least one surface to electromagnetic radiation having a wavelength having a length ranging from about 200 to about 600 nm.
